(19) United States
(12) Patent Application Publication
(10) Pub. No.: US 2024/0366176 A1
(43) Pub. Date: Nov. 7, 2024

(54) SYSTEMS AND METHODS FOR INJECTION DOSE CALIBRATION IN PET IMAGING

(71) Applicant: GE Precision Healthcare LLC, Wauwatosa, WI (US)

(72) Inventors: Yaron Hefetz, Herzeliya (IL); Galit Ankri-Eliahoo, Zichron-Ya'akov (IL); Lilach Shay Levi, Tirat Carmel (IL)

(21) Appl. No.: 18/310,357

(22) Filed: May 1, 2023

(51) Int. Cl. A61B 6/00; A61B 6/03; G16H 10/60

(52) U.S. Cl. CPC A61B 6/582; A61B 6/037; G16H 10/60

(57) ABSTRACT

Methods and systems are herein provided for injection dose calibration in positron emission tomography (PET) imaging. In one example, a method comprises generating one or more calibration images of a region of interest (ROI) of a target anatomy of a patient injected with a radiotracer while the patient is positioned within an imaging system, determining from the ROI of the one or more calibration images a measure of radiotracer uptake within the patient and comparing the determined measure of radiotracer uptake to a reference uptake, outputting a notification of recommendations for modification to one or more initial scan parameters based on the comparison, modifying the one or more initial scan parameters in response to user input, and acquiring diagnostic images of the patient according to the one or more modified scan parameters while the patient is still within the imaging system.

### FIELD

Embodiments of the subject matter disclosed herein relate to medical imaging, and more particularly to calibrating radiotracer injection dose for positron emission tomography imaging.

### BACKGROUND

Medical imaging systems may be used to capture images to assist a physician in making an accurate diagnosis. For example, a physician may use one or more images to visually identify a lesion or other anomalous structure in a patient. As another example, a physician may compare images taken over a series of patient visits to examine the evolution of a structure and/or to evaluate the effectiveness of a treatment. That is, the physician may examine morphological changes, such as changes in size and/or shape, of a lesion to evaluate its characteristics and/or the effectiveness of therapy.

Positron emission tomography (PET) scanning can be used to generate images representing metabolic activity in, for example, a patient. A radioactive tracer, such as Fluorine-18 2-fluoro-2-deoxy-D-glucose (FDG), may be injected into a patient. FDG mimics glucose and, thus, may be taken up and retained by tissues that require glucose for their activities. Tissues with higher metabolic activity will contain more of the tracer. A PET scanner allows detection of the tracer through its radioactive decay. Thus, by detecting and determining the location of the tracer, a PET scanner can be used to generate images representing metabolic activity.

### BRIEF DESCRIPTION

In one example, a method includes generating one or more calibration images of a region of interest (ROI) of a target anatomy of a patient injected with a radiotracer with the patient positioned within an imaging system; determining from the ROI of the one or more calibration images a measure of radiotracer uptake in the patient; comparing the determined measure of radiotracer uptake to a specified reference uptake; outputting a notification to a user, wherein the notification comprises one or more recommendations for modification to one or more initial scan parameters based on comparison between the determined measure of radiotracer uptake and the specified reference uptake; modifying the one or more initial scan parameters in response to user input; and acquiring diagnostic image data of the patient according to the one or more modified scan parameters, wherein the patient is still positioned within the imaging system when the diagnostic image data is acquired.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 is a pictorial view of a multi-modality imaging system according to an embodiment of the invention;

FIG. 2 is a block schematic diagram of an imaging system with a detector, according to an embodiment of the invention;

FIG. 3 is a block schematic diagram of a calibration system of the imaging system of FIG. 2, according to an embodiment of the invention;

FIG. 4 is a flowchart illustrating a method for injection dose calibration, according to an embodiment of the invention;

FIG. 5 is an example image with suboptimal radiotracer uptake due to insufficient radiotracer injection; and

FIG. 6 is an example image without insufficient radiotracer injection.

### DETAILED DESCRIPTION

This description and embodiments of the subject matter disclosed herein relate to methods and systems for calibration of injection dose in Positron Emission Tomography (PET) imaging. In PET imaging, and other imaging modalities herein described a radiotracer (e.g., radioactive tracer, radiopharmaceutical, etc.) is injected into a bloodstream of a patient where uptake of the radiotracer by tissues may be correlated with tissue metabolism. Examples of radiotracers include Fluorine-18 2-fluoro-2-deoxy-D-glucose (FDG), Oxygen-15-H₂O, Carbon-11 choline, Carbon-11 methionine, Fluorine-18 dihydroxyphenylalanine, among others, with FDG being a widely used radiotracer for PET imaging. Metabolically active tissues may uptake the radiotracer and an amount of uptake may be measured by the PET system. There are several methods for measuring the amount of uptake of the radiotracer, including calculating a standardized uptake value (SUV). Specifically, SUV is a ratio of tissue concentration of radiotracer as measured by a PET system and a quotient of decay-corrected amount of injected FDG and a weight of the patient. SUV is provided by equation (1):

$$S U V = \frac{r}{( \frac{{a}^{′}}{w} )}$$

where r is the radiotracer activity concentration (kBq/ml) measured by the PET scanner within a region of interest (ROI), a′ is the decay-corrected amount of injected FDG (or other radiotracer) (kBq), and w is the weight of the patient (g). The weight of the patient may be used as a measure of a distribution volume of the radiotracer. Other measures of radiotracer uptake include relative uptake values, activity concentrations alone (e.g., amount of radioactivity per ml, kBq/ml, not accounting for patient weight or decay-correction), normalized activity concentrations accounting for patient weight (e.g., kBq/(ml/kg)), and dynamic imaging quantitative index values such as Patlak Ki values that represent rate of radiotracer (e.g., FDG) metabolism. Dynamic imaging quantitative index values such as Patlak Ki values may be used with dynamic PET/CT imaging, such as those used for evaluation and/or monitoring of cardiac sarcoidosis.

Calculated and/or measured SUVs, or other measures of radiotracer uptake, may have a large degree of variability due to physical and biological sources of errors. Insufficiencies in injection dose may result from human and/or physical and/or biological sources of error and/or equipment errors. For example, one or more patient parameters, including patient age, patient sex, and patient weight may result in variable uptake of radiotracers even with adequate administration (e.g., injection). Human error, including errors in calculating dose for a patient, inadequate injection (e.g., missing a vein during injection), and others, may result in a lower amount of radiotracer being injected into the patient's bloodstream than expected. Equipment errors may include injector issues, retained radiotracer in tubing, among others, which may also result in a lower amount of radiotracer being injected into the patient's bloodstream than expected. Each of these types of errors may alter an actual decay-corrected amount of injected radiotracer compared to an expected decay-corrected amount of injected radiotracer, thereby falsely increasing or decreasing the calculated uptake value compared to the actual value. Additionally, inconsistent and non-optimized image acquisition, processing, and analysis may result in variable uptake values.

In spite of variable uptake values from patient to patient, scan parameters may remain constant. For example, scan parameters for elderly patients who may not uptake a radiotracer at the same rate as a younger patient are the same as for the younger patient. Additionally, current uptake values may be measured without taking into account the variable sources of error as herein described. In this way, uptake values may be unreliable for diagnostic purposes in the setting of multiple sources of error, both in administration of radiotracer and in uptake calculation as a result of inaccurate administration values.

Systems and methods are disclosed herein for calibration of injection dose in PET imaging. By obtaining one or more calibration images of an ROI of a chosen organ or area of the patient (e.g., a target anatomy), determining an uptake value (e.g., an SUV) of the ROI, and comparing the uptake value to a specified reference uptake value of a set of available uptake values, calibration of scan parameters in light of the injected dose may be performed. For example, if the calculated uptake value for the patient confirms typical uptake for the ROI (e.g., within a specified range of an expected uptake value for the ROI and age/sex of the patient), no change in scan parameters may be made. If the calculated uptake value for the patient is lower or higher than expected uptake value for the ROI (e.g., above or below the range of the specified reference uptake value), one or more recommendations may be outputted for display allowing a user (e.g., a technician or care provider) to select a scan parameter to be modified. Modification of one or more scan parameters may account for variation in radiotracer administration to a patient or uptake by a patient in order to allow for adequate SUV calculation of diagnostic images of the patient by calibration of the injected dose. In this way, the methods provided herein may allow for a sanity check for sufficiency of administered dose and provides the user with options to change scan parameters to account for insufficiencies.

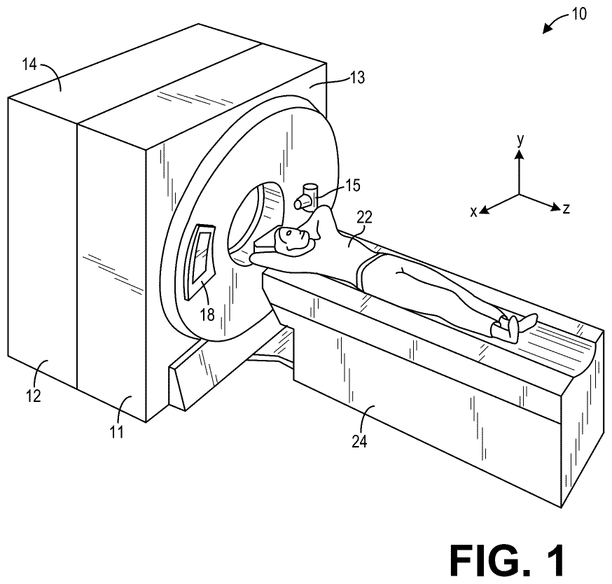

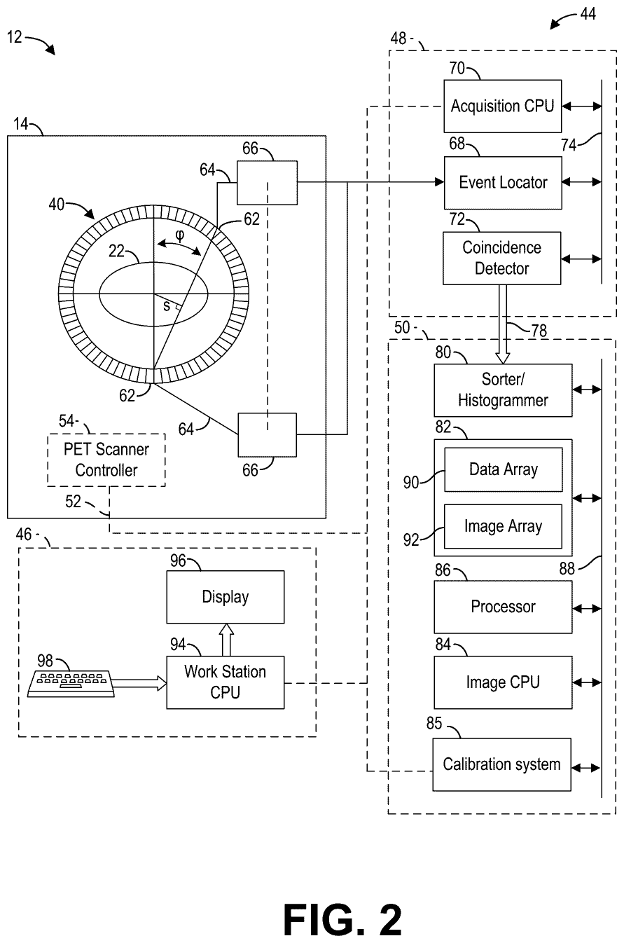

Various embodiments of the invention provide a multi-modality imaging system 10 as shown in FIGS. 1 and 2. Multi-modality imaging system 10 may be any type of imaging system, for example, different types of medical imaging systems, such as a PET, a Single Photon Emission Computed Tomography (SPECT), a Computed Tomography (CT), an ultrasound system, Magnetic Resonance Imaging (MRI), or any other system capable of generating tomographic images. The various embodiments are not limited to multi-modality medical imaging systems, but may be used on a single modality medical imaging system such as a stand-alone PET imaging system or a stand-alone SPECT imaging system, for example. Moreover, the various embodiments are not limited to medical imaging systems for imaging human subjects, but may include veterinary or non-medical systems for imaging non-human objects.

Referring to FIG. 1, the multi-modality imaging system 10 includes a first modality unit 11 and a second modality unit 12. The two modality units enable the multi-modality imaging system 10 to scan an object or patient in a second modality using the second modality unit 12. The multi-modality imaging system 10 allows for multiple scans in different modalities to facilitate an increased diagnostic capability over single-modality systems. In one embodiment, multi-modality imaging system 10 is a Computed Tomography/Positron Emission Tomography (CT/PET) imaging system 10, e.g., the first modality 11 is a CT imaging system 11 and the second modality 12 is a PET imaging system 12. The CT/PET system 10 is shown as including a gantry 13 representative of a CT imaging system and a gantry 14 that is associated with a PET imaging system. As discussed above, modalities other than CT and PET may be employed with the multi-modality imaging system 10.

The gantry 13 includes an x-ray source 15 that projects a beam of x-rays toward a detector array 18 on the opposite side of the gantry 13. Detector array 18 is formed by a plurality of detector rows (not shown) including a plurality of detector elements which together sense the projected x-rays that pass through a medical patient 22. Each detector element produces an electrical signal that represents the intensity of an impinging x-ray beam and hence allows estimation of the attenuation of the beam as it passes through the patient 22. During a scan to acquire x-ray projection data, gantry 13 and the components mounted thereon rotate about a center of rotation.

FIG. 2 is a block schematic diagram of the PET imaging system 12 illustrated in FIG. 1 in accordance with an embodiment of the present invention. The PET imaging system 12 includes a detector ring assembly 40 including a plurality of detector crystals. The PET imaging system 12 also includes a processor or controller 44, to control normalization, image reconstruction processes and perform calibration. Controller 44 is coupled to an operator workstation 46. Controller 44 includes a data acquisition processor 48 and an image reconstruction processor 50, which are interconnected via a communication link 52. PET imaging system 12 acquires scan data and transmits the data to data acquisition processor 48. The scanning operation is controlled from the operator workstation 46. The data acquired by the data acquisition processor 48 is reconstructed using the image reconstruction processor 50.

The detector ring assembly 40 includes a central opening, in which an object or patient, such as patient 22 may be positioned using, for example, a motorized table 24 (shown in FIG. 1). The motorized table 24 is aligned with the central axis of detector ring assembly 40. This motorized table 24 moves the patient 22 into the central opening of detector ring assembly 40 in response to one or more commands received from the operator workstation 46. A PET scanner controller 54, also referred to as the PET gantry controller, is provided (e.g., mounted) within PET system 12. The PET scanner controller 54 responds to the commands received from the operator workstation 46 through the communication link 52. Therefore, the scanning operation is controlled from the operator workstation 46 through PET scanner controller 54.

During operation, when a photon collides with a crystal 62 on a detector ring 40, it produces a scintillation event on the crystal. Each photomultiplier tube or photosensor produces an analog signal that is transmitted on communication line 64 when a scintillation event occurs. A set of acquisition circuits 66 is provided to receive these analog signals. Acquisition circuits 66 produce digital signals indicating the three-dimensional (3D) location and total energy of the event. The acquisition circuits 66 also produce an event detection pulse, which indicates the time or moment the scintillation event occurred. These digital signals are transmitted through a communication link, for example, a cable, to an event locator circuit 68 in the data acquisition processor 48.

The data acquisition processor 48 includes the event locator circuit 68, an acquisition CPU 70, and a coincidence detector 72. The data acquisition processor 48 periodically samples the signals produced by the acquisition circuits 66. The acquisition CPU 70 controls communications on a back-plane bus 74 and on the communication link 52. The event locator circuit 68 processes the information regarding each valid event and provides a set of digital numbers or values indicative of the detected event. For example, this information indicates when the event took place and the position of the scintillation crystal 62 that detected the event. An event data packet is communicated to the coincidence detector 72 through the back-plane bus 74. The coincidence detector 72 receives the event data packets from the event locator circuit 68 and determines if any two of the detected events are in coincidence. Coincidence is determined by a number of factors. First, the time markers in each event data packet must be within a predetermined time period, for example, 12.5 nanoseconds, of each other. Second, the line-of-response (LOR) formed by a straight line joining the two detectors that detect the coincidence event should pass through the field of view in the PET imaging system 12. Events that cannot be paired are discarded. Coincident event pairs are located and recorded as a coincidence data packet that is communicated through a physical communication link 78 to a sorter/histogrammer 80 in the image reconstruction processor 50.

The image reconstruction processor 50 includes the sorter/histogrammer 80. During operation, sorter/histogrammer 80 generates a data structure known as a histogram. A histogram includes a large number of cells, where each cell corresponds to a unique pair of detector crystals in the PET scanner. Because a PET scanner typically includes thousands of detector crystals, the histogram typically includes millions of cells. Each cell of the histogram also stores a count value representing the number of coincidence events detected by the pair of detector crystals for that cell during the scan. At the end of the scan, the data in the histogram is used to reconstruct an image of the patient. The completed histogram containing all the data from the scan is commonly referred to as a “result histogram.” The term “histogrammer” generally refers to the components of the scanner, e.g., processor and memory, which carry out the function of creating the histogram.

The image reconstruction processor 50 also includes a memory module 82, an image CPU 84, an array processor 86, and a communication bus 88. During operation, the sorter/histogrammer 80 counts all events occurring along each projection ray and organizes the events into 3D data. This 3D data, or sinogram, is organized in one exemplary embodiment as a data array 90. Data array 90 is stored in the memory module 82. The communication bus 88 is linked to the communication link 52 through the image CPU 84. The image CPU 84 controls communication through communication bus 88. The array processor 86 is also connected to the communication bus 88. The array processor 86 receives data array 90 as an input and reconstructs images in the form of image array 92. Resulting image arrays 92 are then stored in memory module 82.

The images stored in the image array 92 are communicated by the image CPU 84 to the operator workstation 46. The operator workstation 46 includes a CPU 94, a display 96, and an input device 98. The CPU 94 connects to communication link 52 and receives inputs, e.g., user commands, from the input device 98. The input device 98 may be, for example, a keyboard, mouse, a touch-screen panel, and/or a voice recognition system, and so on. Through input device 98 and associated control panel switches, the operator can control the operation of the PET imaging system 12 and the positioning of the patient 22 for a scan. Similarly, the operator can control the display of the resulting image on the display 96 and can perform image-enhancement functions using programs executed by the workstation CPU 94.

Additionally, as described in greater detail herein, PET imaging system 12 may include a calibration system 85 that may determine if radiotracer uptake is as expected. Via calculation of an uptake value for a specified ROI of a calibration image, the calibration system 85 may determine if radiotracer uptake in the calibration image is an expected uptake (e.g., via comparison of a calculated uptake value of the calibration image to a reference uptake value). Suggestions for scan parameter modifications may be displayed to the operator via workstation CPU 94 and/or display 96. For example, a calibration image of a specified target anatomy, such as a brain, may be taken, a first uptake value may be calculated of the specified target anatomy, and the first uptake value may then be compared to a reference uptake value for the specified organ. The specified organ may be chosen based on uptake consistency. For example, an organ that repeatedly uptakes a radiotracer in predictable amounts may be chosen in order for comparisons to be made to calibrate injection doses. Further, the specified target anatomy may be known as healthy to maintain the uptake consistency. For example, a brain may not be chosen as the specified target tissue in scenarios in which the patient of interest has a disease that would affect uptake in the brain (e.g., Alzheimer's). Reference uptake values may be known or generated for specific patient ages and genders and stored in memory. The reference uptake value may be specific to patient parameters such as patient age, patient sex, and/or patient weight. If the first uptake value deviates from the reference uptake value, the calibration system 85 may output one or more suggested scan parameter modifications to the operator, as described in greater detail below.

The detector ring assembly 40 includes a plurality of detector units. The detector unit may include a plurality of detectors, light guides, scintillation crystals and analog application specific integrated chips (ASICs). For example, the detector unit may include twelve SiPM devices, four light guides, 144 scintillation crystals, and two analog ASICs.

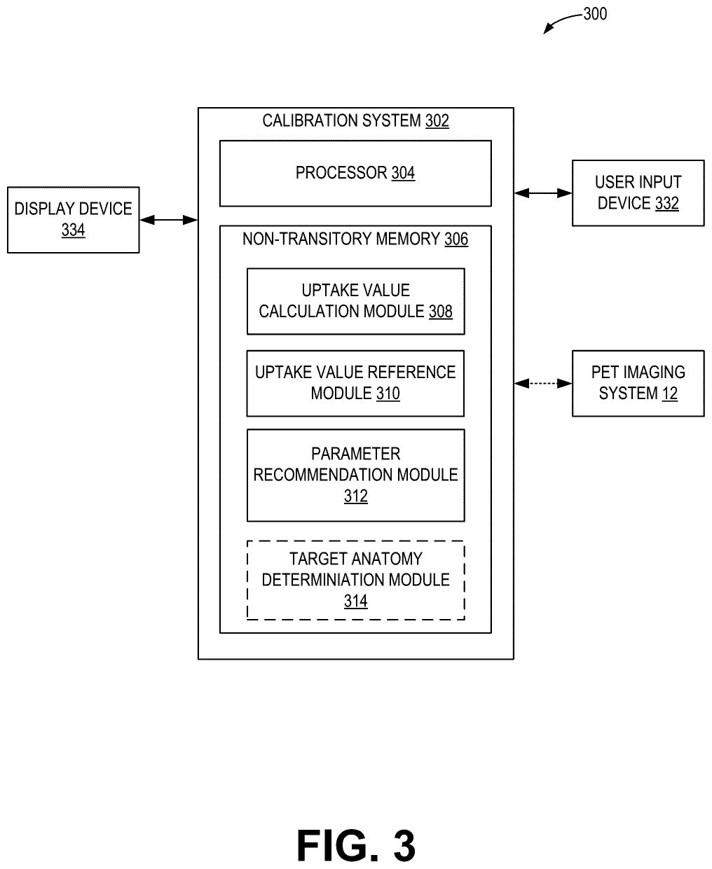

Referring now to FIG. 3, a block diagram 300 shows a prophetic example of a calibration system 302, in accordance with an embodiment, where calibration system 302 may determine if an amount of radiotracer uptake is as expected and, if not, may suggest one or more scan parameter modifications to be used during diagnostic image acquisition performed using a PET imaging system, such as PET imaging system 12 described above in reference to FIGS. 1 and 2. As such, calibration system 302 may be a non-limiting example of calibration system 85 of FIG. 2. In some examples, calibration system 302 may be incorporated into the PET imaging system, as described above. For example, calibration system 302 may rely on controller 44 and memory module 82. In some examples, at least a portion of calibration system 302 is disposed at a device (e.g., workstation, edge device, server, etc.) communicably coupled to the PET imaging system via wired and/or wireless connections, which can receive images from the PET imaging system or from a storage device which stores the images/data generated by the PET imaging system. Calibration system 302 may be operably/communicatively coupled to a user input device 332 and a display device 334. User input device 332 may comprise the input device 98 of the PET imaging system 12, while display device 334 may comprise the display 96 of the PET imaging system 12, at least in some examples.

Calibration system 302 includes a processor 304 configured to execute machine readable instructions stored in non-transitory memory 306. Processor 304 may be single core or multi-core, and the programs executed thereon may be configured for parallel or distributed processing. In some examples, processor 304 may optionally include individual components distributed throughout two or more devices, which may be remotely located and/or configured for coordinated processing. In some examples, one or more aspects of processor 304 may be virtualized and executed by remotely-accessible networked computing devices configured in a cloud computing configuration.

Non-transitory memory 306 may store an uptake value calculation module 308, an uptake value reference module 310, and a parameter recommendation module 312. Uptake value calculation module 308 may include instructions for calculating an uptake value (e.g., an SUV) for an area or region of uptake in an image, either a calibration image or otherwise. In some examples, user input may inform the uptake value calculation module 308 of variables to calculate an uptake value, such as a patient weight, injected dose, and the like as described with reference to equation (1) above.

The uptake value reference module 310 may store a plurality of reference uptake values (e.g., reference SUVs). Each of the plurality of reference uptake values may be specific to a sex and/or age group. Further, the plurality of reference uptake values may be based on the same method as the uptake value calculation module 308. For example, if the uptake value calculation module 308 may calculate a measure of radiotracer uptake as an SUV, the plurality of reference uptake values may be reference SUVs. Additionally, in some examples, ranges of each reference uptake value may also be included. The reference uptake values stored in the uptake value reference module 310 may serve as values for a calculated uptake value to be compared to, wherein the reference uptake values are expected values. The reference uptake values may be sourced from healthy patients and may be of a specified organ or region. For example, the reference uptake values may be of a whole brain, region of a brain, sternum, or other body region/organ. As an example, an uptake value calculated of a region of a calibration image of a patient of interest may be compared to a corresponding reference uptake value of the same sex and age group as the patient of interest.

The parameter recommendation module 312 may comprise one or more scan parameter modifications that may be suggested based on a comparison of a calculated uptake value to a reference uptake value. If the calculated uptake value is determined to deviate from the reference uptake value, the parameter recommendation module 312 may output a notification of one or more suggested scan parameter modifications for display on the display device. The one or more suggested scan parameter modifications may include increasing/decreasing scan duration, changing bed overlap, and/or removing a “hot” location out of a field of view. In some examples, a hot location may result from improper injection, whereby radiotracer remains near an injection site and thus results in increased uptake at the injection site. Increasing/decreasing scan duration may include altering an amount of time taken to acquire a scan. Changing bed overlap may include changing bed positions to increase or decrease amount of overlap between beds. Changing bed overlap may increase or decrease amount of data acquired in the diagnostic scan. Removal of a hot location out of the field of view may change a calculated SUV by removing an area increasing the uptake concentration.

The one or more suggested scan parameter modifications may be based on the calculated uptake value and the comparison between the calculated uptake value and the reference uptake value. For example, an amount of increased scan duration (e.g., in minutes) may be based on amount of deviation of the calculated uptake value compared to the reference uptake value. For example, a first calculated uptake value may be deviated from a first reference uptake value by a first amount. A second calculated uptake value may be deviated from a second reference uptake value by a second amount that is greater than the first amount. Consequently, a first suggested scan duration modification for the first comparison may recommend an increase that is less than an increase recommended by a second suggested scan duration modification for the second comparison.

The one or more suggested scan parameter modifications may be outputted to the display device, as described. Operator input from an operator of the PET imaging system (e.g., a technician or care provider) may indicate to the PET imaging system the modifications that are to be made to scan parameters. For example, the operator may choose to increase scan duration but not change overlap and corresponding operator inputs may indicate to the PET imaging system which modifications are chosen. In some examples, the parameter recommendation module 312 may include specifics for each of the one or more suggested scan parameter modifications that are not explicitly outputted for display to the operator. As an example, a suggestion for increasing scan duration may be outputted for display while specifics of amount of time, such as increasing scan duration by four minutes, may be stored in the parameter recommendation module 312 but not outputted for display. In other examples, all information generated and stored in the parameter recommendation module 312 may be outputted for display.

In some examples, the calibration system 302 may be communicatively coupled to one or more databases, such as an EMR, that stores past medical history data of the patient of interest. In such examples, a target anatomy determination module 314 may obtain data from the one or more databases in order to determine a target anatomy to be used in calibration of injection dose. As described with respect to FIG. 2, a disease or suspicion of disease in a potential target anatomy, such as a degenerative brain disease, may preclude the potential target anatomy, such as a brain, from being chosen as the target anatomy as the disease or suspicion of disease may degrade uptake value results. In some examples, the target anatomy determination module 314 may further include a weighting method whereby potential target anatomies are weighted against one another to determine which of the potential target anatomies may provide a most consistent and/or predictable uptake value for comparison. In this way, processing power and time may be reduced as comparison in uptake values for target anatomies that may produce degraded results may be avoided. Further, time spent in determination may be reduced as compared to manual determination of target anatomy.

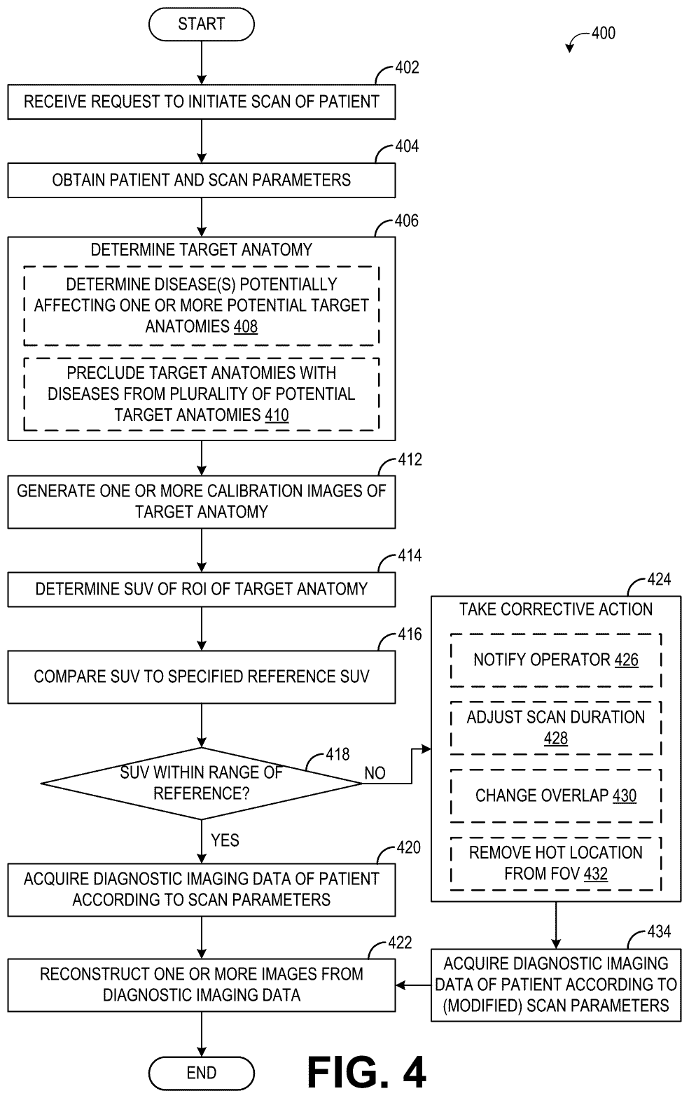

Referring now to FIG. 4, a flowchart illustrating an example method 400 for injection dose calibration is shown. Method 400 may be executable by a calibration system of an imaging system, such as calibration system 302 of FIG. 3 in communication with PET imaging system 12 of FIG. 2. Method 400 may be carried out by one or more processors of a computing device communicatively coupled to the imaging system, such as processor 304 of calibration system 302, according to instructions stored in non-transitory memory, such as non-transitory memory 306 of FIG. 3. It should be understood that while the method 400 is referenced with respect to SUVs, any other reasonable measure of radiotracer uptake, such as dynamic imaging quantitative index values as referenced previously, may be used without departing from the scope of this disclosure.

At 402, method 400 includes receiving a request to initiate a scan of the patient. The request may be a user selection or other user input inputted by a user via a user device (e.g., input device 98 of FIG. 1) in communication with an imaging system, such as PET imaging system 12 of multi-modality imaging system 10 of FIG. 1.

At 404, method 400 includes obtaining patient and scan parameters. One or more patient parameters may include a weight, height, body mass index (BMI), age, sex (e.g., male or female), and/or amount of injected radiotracers, among others, specific to the patient of interest. The one or more patient parameters may be inputted to the PET imaging system by the user via user input to the user device. One or more scan parameters may include scan duration, bed positions, bed overlap, beta value, and the like. In some examples, the one or more scan parameters such as scan duration and bed overlap may be standardized to initial parameters based on a predefined baseline. For example, an initial scan duration, initial field of view, and initial bed overlap may be based on standardized parameters. In other examples, scan parameters may be based on the inputted patient parameters. For example, a scan duration may be dependent on a patient age, sex, and/or weight.

The one or more patient parameters may further define one or more reference SUVs (or other measures of radiotracer uptake) that are to be used in comparison. As described previously, reference SUVs may be specific to patient age, sex, or other patient parameters. Obtaining the patient parameters, as described, may specify a corresponding reference SUV (e.g., a reference SUV specific to a sex and age group that correspond to the inputted patient parameters). Scan parameters may also include scan type, for example any of a whole body PET scan, brain PET scan, cardiac PET scan, and the like. The scan type may be defined by user input as well, and the scan type inputted may indicate a type of reference SUV. For example, a cardiac PET scan may indicate that a reference SUV used may be an SUV of a sternum while a brain PET scan may indicate that a reference SUV used may be an SUV of a brain.

At 406, method 400 includes determining a target anatomy for which calibration images may be generated. The target anatomy may be one of a plurality of potential target anatomies, as described with respect to FIGS. 2 and 3. The potential target anatomies may be organs or regions of the patient's body that uptake radiotracer in a predictable manner, such as a brain or a sternum. In some examples, determination of the target anatomy from the plurality of potential target anatomies may be based on a weighting method, wherein the chosen target anatomy is the target anatomy of the plurality of target anatomies that may provide a most predictable uptake value result for comparison. The target anatomy may be a specified organ or region within the patient. For example, a brain, region of a brain, sternum, or other may be the specified organ or region. The target anatomy may be chosen based on a number of factors contributing to predictability of radiotracer uptake, which may be used as weighting factors in an automatic selection based on data in an electronic medical record, including range of sizes among patients, attenuation, blood supply to the specified organ or region, surrounding tissues hindering image acquisition of the specified organ or region, movement of the region (e.g., periodic movements such as breathing), and the like. The chosen organ or region may be one that is approximately the same size across patients (e.g., has a smaller range of sizes compared to other organs), has little attenuation variation, has minimal movement, and is not surrounded by tissues (e.g., fatty tissue) that may hinder image acquisition. The brain and/or the sternum, for example, having little variability in size, low attenuation variability, and little surrounding tissues, may be chosen as the target anatomy. In an example, the ROI may be selected based on weighting factors, one of which being an amount of processing necessary for monitoring SUV for that particular ROI.

In some examples, disease(s) of the patient that may affect one or more potential target anatomies may be determined, as noted at 408. In such examples, the calibration system may be in communication with one or more databases that store medical history data of the patient. Such medical history data may include data of known or suspected diseases that may affect one or more potential target anatomies. Determination of disease(s) affecting one or more potential target anatomies may preclude target anatomies with diseases from being included in the plurality of potential target anatomies, as noted at 410. Determination of the target anatomy may be an automatic process performed by the calibration system prior to generation of calibration images. Such an approach may utilize stored weighting factors and real-time medical record data for a particular patient so as to automatically select a region of interest for determination of SUV. The automatic selection enables improve use of limited processing resources for determination of SUV for a particular ROI, for example, by avoiding repeat monitoring and further by automatically selecting a ROI that provides a lower processing usage for SUV determination than other viable ROIs for the particular patient, without requiring user input.

At 412, method 400 includes generating one or more calibration images of the target anatomy. The patient may be injected with a radiotracer such as FDG prior to generating the one or more calibration images and the patient may be positioned within the imaging system (e.g., within a gantry of the imaging system) during generation of the one or more calibration images. In some examples, such as when the chosen organ is a brain (e.g., during a head-first positioning scan), the one or more calibration images may be generated while advancing the patient into the gantry. The one or more calibration images may be generated relatively quickly and may not demand diagnostic quality as compared to diagnostic imaging data. The one or more calibration images may be generated of the target anatomy.

The one or more calibration images may be acquired and processed prior to acquisition of final PET data. The one or more calibration images, as will be described herein, may be used to determine whether injected dose of radiotracer and/or uptaken amount of radiotracer is as expected and, if not as expected, the one or more calibration images may be used to suggest scan parameter modifications to calibrate the injected dose.

At 414, an SUV (or other measure of radiotracer uptake) of an ROI of the target anatomy is determined from the one or more calibration images. The SUV may be, as described above, a measure of radiotracer activity within the patient. The ROI of the target anatomy may be, in some examples, a portion of the target anatomy or, in other examples, the entire target anatomy. The determined SUV (e.g., calculated uptake value or calibration uptake value) of the ROI of the target anatomy may be determined based on equation (1) as defined above, wherein radiotracer activity concentration is divided by a ratio of a decay-corrected amount of injected FDG and the patient's weight. The amount of injected FDG and the patient's weight may both be inputted as patient parameters, as noted at 404. The radiotracer activity concentration may be determined by the PET/CT imaging system. The SUV may be a non-zero positive number. In some examples, the calibration SUV may be an average SUV of the target anatomy or an ROI specific SUV of the target anatomy.

At 416, the determined SUV is compared to a specified reference SUV. The specified reference SUV may be a specified reference uptake that is expected for the patient. The specified reference SUV used for comparison may correspond to patient parameters and scan parameters obtained at 404. For example, the specified reference SUV may be specific to sex, age, and scan type. A set of available reference SUVs (and associated ranges for each reference SUV) may be inputted into the calibration system or otherwise known by the calibration system. For example, the set of available reference SUVs may be obtained from an outside source and inputted into the calibration system for use during method 400.

At 418, the method 400 judges whether the determined SUV of the ROI of the target anatomy from the one or more calibration images is within range of the specified reference SUV, wherein the specified reference SUV is specific to the one or more patient parameters, including patient sex and patient age, as well as the target anatomy. In some examples, each reference SUV within the set of available reference SUVs may include a range. The specified reference SUV for the patient (as determined based on patient and scan parameters) may be compared to the determined SUV of the ROI of the target anatomy. If the determined SUV of the ROI of the target anatomy is within the range of the specified reference SUV (YES), method 400 proceeds to 420. If the determined SUV of the ROI of the target anatomy is outside the range of the specified reference SUV (NO), method 400 proceeds to 424.

At 420, diagnostic imaging data of the patient according to the scan parameters is acquired. The scan parameters used to acquire the diagnostic imaging data may be one or more initial scan parameters. Acquisition of diagnostic imaging data may include acquiring image data of the patient, for example including the target anatomy of the patient. In some examples, the diagnostic image data may include regions of the patient's body not included in the one or more calibration images. In other examples, the diagnostic imaging data may include only the same region of the patient's body as the one or more calibration images. The image data may be in the form of a data array that is stored in memory. The diagnostic imaging data may be acquired immediately following determination that the determined SUV is within range of the specified reference SUV, wherein immediately (e.g., in near real-time) denotes that no intentional delay is included and the patient remains within the imaging system (e.g., has not moved from within the imaging system between generation of the one or more calibration images and acquisition of the diagnostic imaging data).

In examples in which the diagnostic imaging data is to be obtained in a head first scan, the one or more calibration images may be generated as the patient is advanced into the gantry of the imaging system as the target anatomy (e.g., a brain) is passed through the gantry during positioning of the patient. The one or more calibration images may be processed to determine the SUV of the ROI of the target anatomy and compare the determined SUV to the reference SUV automatically, semi-automatically, or manually. If within range of the reference SUV, as determined at 418, the diagnostic imaging data may be acquired automatically or semi-automatically in response to this determination and as the one or more calibration images were acquired while advancing the patient into the gantry, an overall spent in confirming injection dose is reduced. 418

At 422, the diagnostic imaging data is reconstructed into one or more images. As is described with reference to FIG. 2, the data array may be reconstructed into one or more images by a reconstruction processor. The one or more images may be three-dimensional or a collection of two-dimensional images. The one or more images may be stored as an image array in memory and may be displayed on a display device for visualization by the user.

At 424, corrective action is to account for the determined SUV being outside the range of the specified reference SUV. In some examples, the corrective action may be taken automatically or semi-automatically in response to determination that the calculated SUV is out of range of the reference SUV. In other examples, notification that corrective action is to be taken may be outputted to the operator, as noted at 426. The notification may include one or more recommendations and may be displayed on the display device of the user input device that is communicatively coupled to the imaging system (e.g., display device 334). User selections via the display device may indicate to the computing device which corrective actions are to be taken, and thus, corrective actions may be taken in response to user input.

Examples of corrective actions may include, in some examples, modifications based on the one or more recommendations that generate one or more modified scan parameters. Corrective actions may include modifying scan duration as noted at 428, modifying bed overlap as noted at 430, and removing hot location(s) from a field of view as noted at 432. Modifying scan duration may include increasing or decreasing duration of scan acquisition. Modifying bed overlap may increasing or decreasing amount of overlap between adjacent beds to increase or decrease acquired data. Removing a hot location from the field of view may include adjusting the patient's position within the imaging system or adjusting an orientation and/or position of the field of view such that the hot location is no longer imaged.

Recommended modifications to the one or more initial scan parameters, as outputted to the user, may be dependent upon the determined SUV of the ROI of the target anatomy and/or the comparison between the determined SUV and the specified reference SUV. For example, a determined SUV that is lower than a specified reference SUV may indicate an increased scan duration and/or an increased overlap. Increased scan duration increases radiotracer activity detection within the ROI and increased overlap may increase amount of data acquired, thereby increasing SUV. As another example, a determined SUV that is higher than a specified reference SUV may indicate decreased scan duration, decreased overlap, and/or removal of a hot location from the field of view. Hot locations within the field of view may increase an average calculated SUV and as such, removal of the hot location from the field of view may decrease calculated SUV. The corrective actions taken may allow for calibration of injection dose, whereby scan parameters are calibrated to compensate for insufficient injection or uptake of the radiotracer.

At 434, diagnostic imaging data is acquired of the patient according to the one or more modified scan parameters. Based on the scan parameter modifications as issued by the corrective actions taken at 422, for example with one of a modified scan time, modified bed overlap, and/or modified field of view, diagnostic imaging data may be acquired. SUVs of the diagnostic imaging data may be appropriately adjusted based on the modifications to the scan parameters when compared to the calibration SUV from the one or more calibration images. In this way, the one or more calibration images and comparison between a determined SUV of the one or more calibration images and a specified reference SUV may allow for consideration and compensation of any errors in radiotracer administration or variations in radiotracer uptake/metabolism. Similar to as described above, the diagnostic imaging data may be acquired immediately following modification of the one or more initial scan parameters.

Corrective actions may be taken while the patient remains within the imaging system such that a workflow for generating the calibration images, modifying scan parameters, and acquiring diagnostic imaging data is completed while the radiotracer is still actively being taken up by the patient. In this way, calibration of injected dose of radiotracer may be performed in near real-time, thereby allowing the diagnostic imaging data to be acquired while the radiotracer within the patient is still actively being taken up by the patient. Thus, the diagnostic imaging data may be acquired even when injected dose of radiotracer is suboptimal as compensation for suboptimal injection may be performed in real-time. Further, processing demands of the imaging system may be reduced, as compensation of suboptimal radiotracer administration or uptake may be performed without having to repeat scan acquisition and/or radiotracer administration, therefore saving time for both the patient and users of the imaging system.

Following acquisition of the diagnostic imaging data, one or more images may be reconstructed from the diagnostic imaging data. The one or more images and/or the diagnostic imaging data may be saved and stored to memory of the computing device. Further, the one or more images may be outputted for display on the display device communicatively coupled to the computing device.

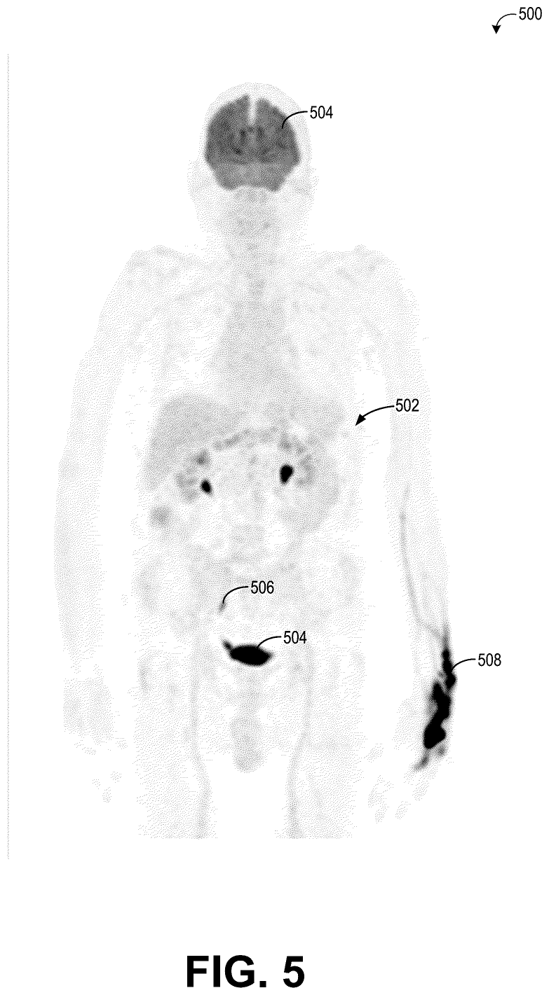

Referring now to FIG. 5, an example of a first image 500 is shown. The first image 500 as shown in FIG. 5 may be a two-dimensional projected rendering, such as a maximum contrast projection (MIP) image, of one or more images of a patient. First image 500 and/or one or more images used to generate the first image 500 may be of a patient acquired with an imaging system, such as PET imaging system 12 of FIG. 1, according to one or more scan parameters. The scan parameters, as discussed above, may include bed overlaps, bed position, scan duration, and the like. The first image 500 as shown may result from insufficient injection of radiotracer, as will be further described.

The first image 500 may depict one or more areas of radiotracer uptake within the patient, wherein the radiotracer was injected into the patient prior to diagnostic data acquisition. A plurality of uptake regions 502 may be seen within the first image 500. The plurality of uptake regions 502 may include one or more expected uptake regions 504 as well as one or more suspicious uptake regions 506. The one or more expected uptake regions 504 may be regions of patient anatomy known to uptake the radiotracer in predictable ways. For example, a brain and/or a bladder may be organs that uptake radiotracer in predictable ways when healthy. The one or more suspicious uptake regions 506 may be regions not expected to uptake radiotracer and are thus considered suspicious for a disease process such as malignancy.

Further, the plurality of uptake regions 502 may also include one or more artifact uptake regions 508. In some examples, the one or more artifact uptake regions 508 may result from insufficient radiotracer injection. As an example, injection of radiotracer partially outside a vein may result in retained radiotracer near an injection site. The retained radiotracer, as depicted in FIG. 5, may remain near the injection site and therefore the retained radiotracer may not circulate through the patient's bloodstream. As a result of retained radiotracer near the injection site, an actual decay-corrected amount of injected radiotracer may be lower compared to an expected decay-corrected amount of injected radiotracer. The expected decay-corrected amount of injected radiotracer may be used to calculate an uptake value (e.g., an SUV) and therefore the calculated uptake value may be lower than expected.

In examples in which insufficient injection occurs, calibration of injection dose may be performed in order to compensate for the insufficiency as described with respect to FIG. 4. In this way, when insufficient injection or uptake occur, by comparing a calculated uptake value in a calibration image and adjusting the one or more scan parameters accordingly, calculated uptake values of the final diagnostic data may be calibrated to the insufficient injection or uptake, thus reducing variability in uptake value calculation.

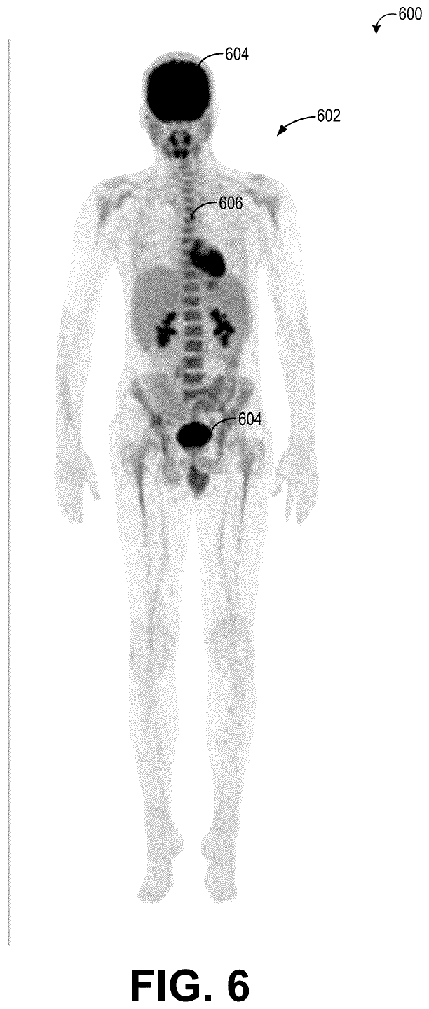

Turning to FIG. 6, an example second image 600 is shown. The second image 600, similar to the first image 500, may be a two-dimensional projected rendering, such as a MIP image, of one or more images of a patient. Second image 600 and/or the one or more images used to generate the second image 600 may be of a patient acquired with an imaging system, such as PET imaging system 12 of FIG. 1, according to one or more scan parameters. The scan parameters, as discussed above, may include bed overlaps, bed position, scan duration, and the like.

The second image 600 may depict one or more areas of radiotracer uptake within the patient, wherein the radiotracer was injected into the patient prior to diagnostic data acquisition. A plurality of uptake regions 602 may be seen within the second image 600. The plurality of uptake regions 602 may include one or more expected uptake regions 604 as well as one or more suspicious uptake regions 606. The one or more expected uptake regions 604, as discussed above, may be regions of patient anatomy known to uptake the radiotracer in predictable ways. For example, a brain and/or a bladder may be organs that uptake radiotracer in predictable ways when healthy. The one or more suspicious uptake regions 606 may be regions not expected to uptake radiotracer and are thus considered suspicious for a disease process such as malignancy.

In comparison to the first image 500, second image 600 does not include artifact uptake regions. In this way, in some examples, during comparison of a calculated uptake value in a calibration image to a reference uptake value, as is described with respect to method 400, the calculated uptake value may be within range of the reference uptake value. Determination that the calculated uptake value is within range of the reference uptake value may indicate that the injected dose of radiotracer is sufficient and as such no change in one or more scan parameters may be recommended or executed.

A technical effect of the systems and methods herein disclosed is that inaccuracies in measured uptake values as a result of insufficiencies and/or variability in radiotracer administration or uptake may be compensated for by calibration of injection dose. As discussed, insufficiencies and/or errors in radiotracer administration or inputted patient parameters, or variability in individual patient uptake of administered radiotracer may result in inaccuracies in calculated uptake values of PET image data. A sanity check, by way of calculating a calibration uptake value of a calibration image of a patient and comparing the calibration uptake value to a reference uptake value that corresponds to sex and age of the patient, may be completed. The sanity check may allow for determination of whether the calibration uptake value is as expected. If not as expected, one or more modifications to scan parameters may be implemented.

The modifications recommended may compensate for variability in radiotracer administration or uptake in near real-time, thereby allowing for calibration of the injected dose and resulting in increased consistency of uptake value calculation. Further, implementation of recommended modifications automatically or semi-automatically may reduce time spent in calibration as well as reducing processing power. As the calibration images are generated with the patient in the imaging system and the diagnostic imaging data is obtained with the patient still within the imaging system, having not moved from within the imaging system between, time spent in calibration and/or compensation may be reduced.

Compensating for suboptimal radiotracer uptake either as a result of variability in patient uptake or human and/or injector error may reduce further processing demands of the imaging system by way of reducing demand for repeat scans and/or image acquisitions. Further, increased consistency of uptake value calculation may allow for consistency of use and/or indicated metabolic activity in clinical and/or diagnostic settings. In this way, increased accuracy of uptake value calculation may be achieved, thereby allowing the calculated uptake values to be used for diagnostic purposes by a care provider.

The disclosure also provides support for a method, comprising: generating one or more calibration images of a region of interest (ROI) of a target anatomy of a patient injected with a radiotracer with the patient positioned within an imaging system, determining from the ROI of the one or more calibration images a measure of radiotracer uptake in the patient, comparing the determined measure of radiotracer uptake to a specified reference uptake, outputting a notification to a user, wherein the notification comprises one or more recommendations for modification to one or more initial scan parameters based on comparison between the determined measure of radiotracer uptake and the specified reference uptake, modifying the one or more initial scan parameters in response to user input, and acquiring diagnostic image data of the patient according to the one or more modified scan parameters, wherein the patient is still positioned within the imaging system when the diagnostic image data is acquired. In a first example of the method, modification of the one or more initial scan parameters comprises corrective action to the one or more initial scan parameters, wherein the corrective action includes at least one of adjusting scan duration, changing bed overlap, and removing a hot location from a field of view. In a second example of the method, optionally including the first example, the method further comprises: obtaining the one or more initial scan parameters and one or more patient parameters, wherein the one or more patient parameters include patient sex and age. In a third example of the method, optionally including one or both of the first and second examples, the one or more initial scan parameters include scan duration, bed overlap, and scan type. In a fourth example of the method, optionally including one or more or each of the first through third examples, the specified reference uptake corresponds to the one or more patient parameters. In a fifth example of the method, optionally including one or more or each of the first through fourth examples, the target anatomy is determined from a plurality of potential target anatomies based on predictability of radiotracer uptake. In a sixth example of the method, optionally including one or more or each of the first through fifth examples, the measure of radiotracer uptake and the specified reference uptake are one of standardized uptake values (SUVs) and normalized activity concentrations (e.g., accounting for patient weight).

The disclosure also provides support for a system, comprising: a computing device communicatively coupled to a positron emission tomography (PET) system configured to image a patient, the computing device configured with instructions in non-transitory memory that when executed cause the computing device to: obtain one or more patient parameters of the patient and one or more initial scan parameters of the PET system, obtain one or more calibration images of a target anatomy of the patient with the PET system, calculate a standardized uptake value (SUV) of a region of interest (ROI) of the target anatomy within the one or more calibration images, compare the calculated SUV to a specified reference SUV, determine if the calculated SUV is within range of the specified reference SUV, in response to a determination that the calculated SUV is out of range of the specified reference SUV, automatically modify the one or more initial scan parameters into one or more modified scan parameters, and obtain diagnostic imaging data with the PET system according to the one or more modified scan parameters. In a first example of the system, the computing device is further configured with instructions that when executed cause the computing device to output a notification to an operator of the PET system, wherein the notification indicates one or more recommendations for modification to the one or more initial scan parameters in response to determination that the calculated SUV is out of range of the specified reference SUV, and in response to user input, modifying the one or more initial scan parameters into the one or more modified scan parameters. In a second example of the system, optionally including the first example, modifying the one or more initial scan parameters comprises changing bed overlap, changing scan duration, and removing a hot location from a field of view of the PET system. In a third example of the system, optionally including one or both of the first and second examples, the computing device is further configured with instructions that when executed cause the computing device to automatically initiate acquisition of diagnostic imaging data according to the one or more initial scan parameters in response to determination that the calculated SUV is within range of the specified reference SUV. In a fourth example of the system, optionally including one or more or each of the first through third examples, the computing device is further configured with instructions that when executed cause the computing device to reconstruct the diagnostic imaging data into one or more images, save the one or more images to memory, and output the one or more images for display on a display device communicatively coupled to the computing device. In a fifth example of the system, optionally including one or more or each of the first through fourth examples, the calculated SUV is calculated based on one of an average SUV of the target anatomy and an ROI specific SUV of the target anatomy. In a sixth example of the system, optionally including one or more or each of the first through fifth examples, the target anatomy is one of a whole brain, a region of a brain, and a sternum, and wherein the target anatomy is automatically selected based on stored weighting factors and real time medical record data. In a seventh example of the system, optionally including one or more or each of the first through sixth examples, the specified reference SUV corresponds to the one or more patient parameters and the one or more patient parameters comprise patient age and patient sex and wherein the specified reference SUV is stored in memory of the computing device.

The disclosure also provides support for a method for a positron emission tomography (PET) system sanity check, comprising: obtaining a calibration image of a brain of a patient, calculating a calibration uptake value of the brain of the patient based on the calibration image, comparing the calibration uptake value to a reference uptake value, wherein the reference uptake value is specific to age and sex of the patient, notifying an operator of the PET system of recommendations for modification to initial scan parameters based on comparison between the calibration uptake value and the reference uptake value, modifying at least one of an initial scan duration, an initial bed overlap, and an initial field of view position based on comparison between the calibration uptake value and the reference uptake value in response to operator input, and obtaining diagnostic imaging data according to modified scan parameters, wherein the modified scan parameters comprise at least one of a modified scan duration, a modified bed overlap, and a modified field of view. In a first example of the method, the modified field of view comprises removal of a hot location from the initial field of view. In a second example of the method, optionally including the first example, the method further comprises: obtaining the diagnostic imaging data according to initial scan parameters in response to determination that the calibration uptake value is within range of the reference uptake value. In a third example of the method, optionally including one or both of the first and second examples, the calibration image is obtained according to the initial scan parameters. In a fourth example of the method, optionally including one or more or each of the first through third examples, the calibration uptake value is one of a standardized uptake value (SUV) and a normalized activity concentration (e.g., accounting for patient weight).

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
generating one or more calibration images of a region of interest (ROI) of a target anatomy of a patient injected with a radiotracer with the patient positioned within an imaging system;
determining from the ROI of the one or more calibration images a measure of radiotracer uptake in the patient;
comparing the determined measure of radiotracer uptake to a specified reference uptake;
outputting a notification to a user, wherein the notification comprises one or more recommendations for modification to one or more initial scan parameters based on comparison between the determined measure of radiotracer uptake and the specified reference uptake;
modifying the one or more initial scan parameters in response to user input; and
acquiring diagnostic image data of the patient according to the one or more modified scan parameters, wherein the patient is still positioned within the imaging system when the diagnostic image data is acquired.

2. The method of claim 1, wherein modification of the one or more initial scan parameters comprises corrective action to the one or more initial scan parameters, wherein the corrective action includes at least one of adjusting scan duration, changing bed overlap, and removing a hot location from a field of view.

3. The method of claim 1, further comprising obtaining the one or more initial scan parameters and one or more patient parameters, wherein the one or more patient parameters include patient sex and age.

4. The method of claim 3, wherein the one or more initial scan parameters include scan duration, bed overlap, and scan type.

5. The method of claim 3, wherein the specified reference uptake corresponds to the one or more patient parameters.

6. The method of claim 1, wherein the target anatomy is determined from a plurality of potential target anatomies based on predictability of radiotracer uptake.

7. The method of claim 1, wherein the measure of radiotracer uptake and the specified reference uptake are one of standardized uptake values (SUVs) and normalized activity concentrations.

8. A system, comprising:
a computing device communicatively coupled to a positron emission tomography (PET) system configured to image a patient, the computing device configured with instructions in non-transitory memory that when executed cause the computing device to:
obtain one or more patient parameters of the patient and one or more initial scan parameters of the PET system;
obtain one or more calibration images of a target anatomy of the patient with the PET system;
calculate a standardized uptake value (SUV) of a region of interest (ROI) of the target anatomy within the one or more calibration images;
compare the calculated SUV to a specified reference SUV;
determine if the calculated SUV is within range of the specified reference SUV;
in response to a determination that the calculated SUV is out of range of the specified reference SUV, automatically modify the one or more initial scan parameters into one or more modified scan parameters; and
obtain diagnostic imaging data with the PET system according to the one or more modified scan parameters.

9. The system of claim 8, wherein the computing device is further configured with instructions that when executed cause the computing device to output a notification to an operator of the PET system, wherein the notification indicates one or more recommendations for modification to the one or more initial scan parameters in response to determination that the calculated SUV is out of range of the specified reference SUV; and in response to user input, modifying the one or more initial scan parameters into the one or more modified scan parameters.

10. The system of claim 9, wherein modifying the one or more initial scan parameters comprises changing bed overlap, changing scan duration, and removing a hot location from a field of view of the PET system.

11. The system of claim 8, wherein the computing device is further configured with instructions that when executed cause the computing device to automatically initiate acquisition of diagnostic imaging data according to the one or more initial scan parameters in response to determination that the calculated SUV is within range of the specified reference SUV.

12. The system of claim 11, wherein the computing device is further configured with instructions that when executed cause the computing device to reconstruct the diagnostic imaging data into one or more images, save the one or more images to memory, and output the one or more images for display on a display device communicatively coupled to the computing device.

13. The system of claim 8, wherein the calculated SUV is calculated based on one of an average SUV of the target anatomy and an ROI specific SUV of the target anatomy.

14. The system of claim 8, wherein the target anatomy is one of a whole brain, a region of a brain, and a sternum, and wherein the target anatomy is automatically selected based on stored weighting factors and real time medical record data.

15. The system of claim 8, wherein the specified reference SUV corresponds to the one or more patient parameters and the one or more patient parameters comprise patient age and patient sex and wherein the specified reference SUV is stored in memory of the computing device.

16. A method for a positron emission tomography (PET) system sanity check, comprising:
obtaining a calibration image of a brain of a patient;
calculating a calibration uptake value of the brain of the patient based on the calibration image;
comparing the calibration uptake value to a reference uptake value, wherein the reference uptake value is specific to age and sex of the patient;
notifying an operator of the PET system of recommendations for modification to initial scan parameters based on comparison between the calibration uptake value and the reference uptake value;
modifying at least one of an initial scan duration, an initial bed overlap, and an initial field of view position based on comparison between the calibration uptake value and the reference uptake value in response to operator input; and
obtaining diagnostic imaging data according to modified scan parameters, wherein the modified scan parameters comprise at least one of a modified scan duration, a modified bed overlap, and a modified field of view.

17. The method of claim 16, wherein the modified field of view comprises removal of a hot location from the initial field of view.

18. The method of claim 16, further comprising obtaining the diagnostic imaging data according to initial scan parameters in response to determination that the calibration uptake value is within range of the reference uptake value.

19. The method of claim 16, wherein the calibration image is obtained according to the initial scan parameters.

20. The method of claim 16, wherein the calibration uptake value is one of a standardized uptake value (SUV) and a normalized activity concentration.
